(19) United States
(12) Patent Application Publication
(10) Pub. No.: US 2026/0115189 A1
(43) Pub. Date: Apr. 30, 2026

(54) Combination Treatment Regimens - SMARCA2 Degrader With Taxane Anticancer Agent

(71) Applicant: PRELUDE THERAPEUTICS INCORPORATED, Wilmington, DE (US)

(72) Inventors: Koichi Ito, Philadelphia, PA (US); Michael Hulse, Doylestown, PA (US); Peggy Scherle, Wayne, PA (US); Krishna Vaddi, Chadds Ford, PA (US)

(21) Appl. No.: 18/906,385

(22) Filed: Oct. 4, 2024

Related U.S. Application Data
(60) Provisional application No. 63/587,767, filed on Oct. 4, 2023.
(60) Provisional application No. 63/594,108, filed on Oct. 30, 2023.
(60) Provisional application No. 63/566,486, filed on Mar. 18, 2024.
(60) Provisional application No. 63/662,462, filed on Jun. 21, 2024.

(51) Int. Cl. A61K 31/5025; A61K 31/337; A61K 45/06; A61P 35/00

(52) U.S. Cl. CPC A61K 31/5025; A61K 31/337; A61K 45/06; A61P 35/00

(57) ABSTRACT

Disclosed are methods of treating cancer comprising administering to a subject a treatment regimen comprising a compound of Formula (I):

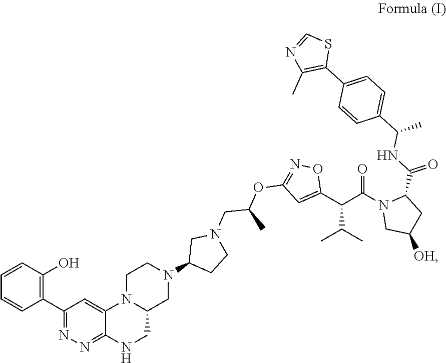 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof, and a taxane anticancer agent.

### CROSS-REFERENCE TO RELATED APPLICATIONS

This application claims the benefit of U.S. Provisional Application No. 63/587,767, filed Oct. 4, 2023; U.S. Provisional Application No. 63/594,108, filed Oct. 30, 2023; U.S. Provisional Application No. 63/566,486, filed Mar. 18, 2024, and U.S. Provisional Application No. 63/662,462, filed Jun. 21, 2024. Each of the aforementioned applications is incorporated by reference herein in its entirety.

### TECHNICAL FIELD

The disclosure pertains to methods of treating cancer comprising administering to a subject a treatment regimen comprising a SMARCA2 degrader and a taxane anticancer agent.

### BACKGROUND

SMARCA2 and SMARCA4 are the core catalytic subunits of the SWI/SNF complexes, which play an important role in controlling gene expression by remodeling chromatin. SMARCA4 is mutated in multiple cancers and SMARCA4-deficient cancer cells can become highly dependent on SMARCA2 for their survival. Therefore, targeting SMARCA2 in SMARCA4-deleted cancers using selective SMARCA2 degraders induces synthetic lethality, while sparing SMARCA4 wild-type normal cells. Cell lines with SMARCA4 damaging mutation or low expression show high SMARCA2 gene dependency scores, suggesting the synthetic lethal relationship of targeting SMARCA2 and SMARCA4-deficiency. SMARCA4 mutations occur in different types of cancer including lung adenocarcinoma, skin cancer/melanoma, uterine/endometrioid adenocarcinoma, bladder cancer, colon adenocarcinoma, among others. In addition to damaging mutation (“loss of SMARCA4 protein”), some tumors express SMARCA4 hotspot missense mutations near its ATP binding site or DNA binding site that likely alters biological function of SMARAC4.

The compound of Formula (I) is a SMARCA2 degrader:

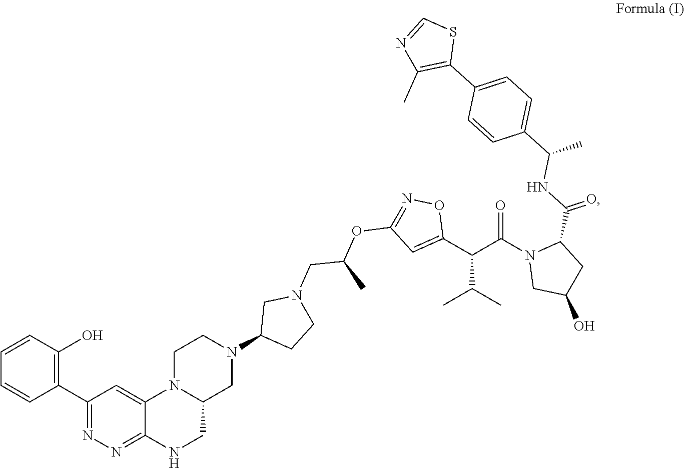 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

that was disclosed in WO2021252666. Formula (I) regulates cancer cell proliferation and growth through cell cycle arrest and DNA replication inhibition in SMARCA4 mutated cancer cells.

Docetaxel is a taxane anticancer agent that is known to regulate cell division by binding to microtubules and inhibiting its polymerization dynamics in cancer cells. See Pienta, K. J., Preclinical mechanisms of action of docetaxel and docetaxel combinations in prostate cancer. Semin Oncol, 2001. 28(4 Suppl 15): p. 3-7. Other studies also demonstrate that docetaxel induces mitochondria priming and sensitizes cancer cells to apoptosis. See Ni Chonghaile, T., et al., Pretreatment mitochondrial priming correlates with clinical response to cytotoxic chemotherapy. Science, 2011. 334(6059): p. 1129-33.

### SUMMARY

The disclosure provides methods of treating cancer in a subject in need thereof, comprising administering to the subject a treatment regimen comprising:
(a) a compound of Formula (I):

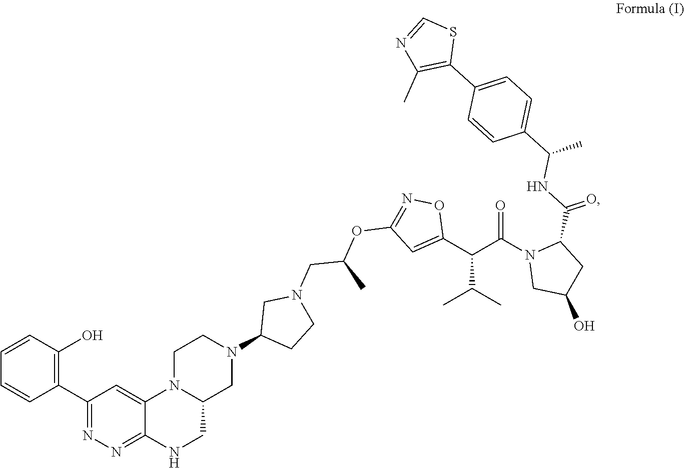 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof; and
(b) a taxane anticancer agent.

In some aspects, the cancer is a SMARCA4-deleted cancer.

### BRIEF DESCRIPTION OF THE DRAWINGS

FIGS. 1A and 1B show Formula (I)+docetaxel combination synergy evaluated by CTG assay and SynergyFinder. In the SMARCA4-low expressing NSCLC cell line NCI-H2030, cells were dosed with Formula (I) and docetaxel in a synergy matrix and incubated for 7 days. Following this, % cell viability was calculated by normalizing the chemiluminescence intensity of each well to the DMSO control wells (0 dose) on the same plate. See FIG. 1A. SynergyFinder was used to calculate ZIP scores (FIG. 1B).

FIGS. 2A-2D show Formula (I)+docetaxel combination effects on the cell cycle were evaluated in the SMARCA4-del NSCLC cell line NCI-H838 by flow cytometry. In the SMARCA4-del NSCLC cell line NCI-H838, cells were dosed with Formula (I) and docetaxel and incubated for 48 hrs. Following this, cells were incubated with EdU, fixed, permeabilized and stained with DRAQ5™ prior to cell cycle analysis. FIG. 2A shows Formula (I) single agent; FIG. 2A shows Docetaxel single agent; FIG. 2C shows 20 nM Formula (I)+docetaxel; FIG. 2D shows 100 nM Formula (I)+docetaxel.

FIGS. 3A-3E show that the Formula (I) combination with docetaxel resulted in deeper tumor growth inhibition (TGI) compared to each single agent alone in the SMARCA4-deficient NCI-H838 Lung Cancer Xenograft Model in Mice. NOD/SCID female mice were inoculated subcutaneously with NCI-H838-#5 cells (5×10⁶) into the right flank in PBS 0.1 ml mixed with Matrigel. When tumor volumes reached approximately 100-150 mm³, mice were randomized and dosed with Formula (I) at 100 mg/kg s.c. Q3D, docetaxel 10 mg/kg on day 0 and day 7, 5 mg/kg on day 14 and day 21 i.v. QW or Formula (I) at 100 mg/kg s.c. Q3D+docetaxel 5/10 mg/kg i.v. QW (FIG. 3A). Body weight changes in these studies are shown in FIG. 3B. FIG. 3C shows tumor volume of NCI-H838 in mice dosed with Formula (I) 75 mg/kg i.v. QW and docetaxcel 10 mg/kg on day 0 and day 7 i.v. FIG. 3D shows tumor volume of NCI-H838 in mice dosed with Formula (I) 75 mg/kg i.v. QW and docetaxcel 10 mg/kg Q3W i.v. FIG. 3E shows tumor volume of NCI-H838 in mice dosed with Formula (I) 75 mg/kg i.v. QW and docetaxcel 25 mg/kg Q3W.* P<0.05 ** P<0.05, *** P<0.001 versus vehicle (two-tailed unpaired Mann-Whitney test).

FIGS. 4A and 4B show that the Formula (I) combination with nab-paclitaxel resulted in deeper tumor growth inhibition (TGI) compared to each single agent alone in the SMARCA4-deficient NCI-H838 Lung Cancer Xenograft Model in Mice. NOD/SCID female mice were inoculated subcutaneously with NCI-H838-#5 cells (5×10⁶) into the right flank in PBS 0.1 ml mixed with Matrigel. When tumor volumes reached approximately 100-150 mm³, mice were randomized and dosed with Formula (I) at 50 or 75 mg/kg i.v. QW, nab-paclitaxel 30 mg/kg i.v. Q3W or Formula (I) at 50 or 75 mg/kg i.v. QW+nab-paclitaxel 30 mg/kg i.v. Q3W (FIG. 4A). Body weight changes in these studies are shown in FIG. 4B.

FIG. 5 shows SMARCA2 protein levels are abolished in tumors by Formula (I) treatment but unaffected by docetaxel treatment. Tumor tissues collected 4 hr and 24 hr post final dosing of Formula (I) 100 mg/kg s.c. Q3D, docetaxel 10/5 mg/kg i.v. QW and Formula (I) 100 mg/kg s.c. Q3D+docetaxel 10/5 mg/kg i.v. QW dosing regimens. Tumor tissues were analyzed for SMARCA2 protein levels by semi-quantitative western blot.

FIGS. 6A and 6B show Formula (I) combination with docetaxel resulted in deeper tumor growth inhibition (TGI) compared to each single agent alone in the SMARCA4-deficient A549 Lung Cancer Xenograft Model in Mice. BALB/c Nude mice were inoculated subcutaneously with A549 tumor cells (5×10⁶). When tumor volumes reached 150-200 mm³, mice were randomized and dosed with Formula (I) at 200 mg/kg s.c. Q3D, docetaxel May 10, 2020 mg/kg i.v. QW or Formula (I) at 200 mg/kg s.c. Q3D+docetaxel May 10, 2020 mg/kg i.v. QW (FIG. 6A). Body weight changes in these studies are shown in FIG. 6B. *P<0.05 ** P<0.05, *** P<0.001 versus vehicle (two-tailed unpaired Mann-Whitney test).

FIGS. 7A and 7B show Formula (I) combination with docetaxel resulted in no significant increase in growth inhibition (TGI) compared to each single agent alone in the SMARCA4-WT Calu-6 Lung Cancer Xenograft Model in Mice. BALB/c Nude mice were inoculated subcutaneously with Calu-6 tumor cells (10×10⁶). When tumor volumes reached 150-200 mm³, mice were randomized and dosed with Formula (I) at 100 mg/kg s.c. Q3D, docetaxel 5/10 mg/kg i.v. QW or Formula (I) at 100 mg/kg s.c. Q3D+docetaxel 5/10 mg/kg i.v. QW (FIG. 7A). Body weight changes in these studies are shown in FIG. 7B. * P<0.05 ** P<0.05, *** P<0.001 versus vehicle (two-tailed unpaired Mann-Whitney test).

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination.

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

The articles “a,” “an,” and “the” as used herein and in the appended claims are used herein to refer to one or to more than one (e.g., to at least one) of the grammatical object of the article unless the context clearly indicates otherwise. By way of example, “an element” means one element or more than one element.

Where a range of values is provided, it is to be understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

When a range of values is expressed, it is to be understood that the explicitly stated upper and lower limits of the range and all intervening individual values, and all intervening subranges are encompassed by the range. All ranges are inclusive and combinable.

When values are expressed herein as approximations, such as by use of the antecedent “about,” it is understood that the particular value forms another embodiment. In some embodiments, “about” refers to a value with ±10% of the particular value.

The term “administering,” when used in the context of administering a therapeutic agent to a subject, refers to introducing the therapeutic agent into the subject's body. For example, therapeutic agents may be introduced into a subject's body orally, nasally, subcutaneously, intravenously, intravesically, intramuscularly, transdermally, vaginally, rectally or in any combination thereof.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The term “subject” is used herein to describe an animal, for example, a mammal, to whom treatment with the treatment regimen according to the disclosure is provided. In some aspects, the mammal is a human to whom treatment is provided. In other aspects, the mammal is a non-human to whom treatment is provided.

“Pharmaceutically acceptable” means approved or approvable by a regulatory agency of the United States Federal government or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethane-sulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. See for example, Remington, J. P. (2020). Remington, the science and practice of pharmacy, Elsevier Science.

As used herein, the term “treatment regimen” refers to coordinated dosages and administration timings of each of Formula (I) (or pharmaceutically acceptable salt thereof) and the taxane anticancer agent such that Formula (I) (or pharmaceutically acceptable salt thereof) and the taxane anticancer agent are administered to the subject during an overlapping period of time. Thus, the “treatment regimen” of the disclosure encompasses both concurrent administration of the compound of Formula (I) (or a pharmaceutically acceptable salt thereof) and the taxane anticancer agent; and sequential administration of the compound of Formula (I) (or a pharmaceutically acceptable salt thereof) and a taxane anticancer agent. Moreover, the treatment regimen encompasses administration of the compound of Formula (I) (or a pharmaceutically acceptable salt thereof) and a taxane anticancer agent on the same day; on different days; and any permutation thereof.

In some aspects, the disclosure is directed to methods of treating cancer in a subject in need thereof, comprising administering to the subject a treatment regimen comprising:
(a) a compound of Formula (I):

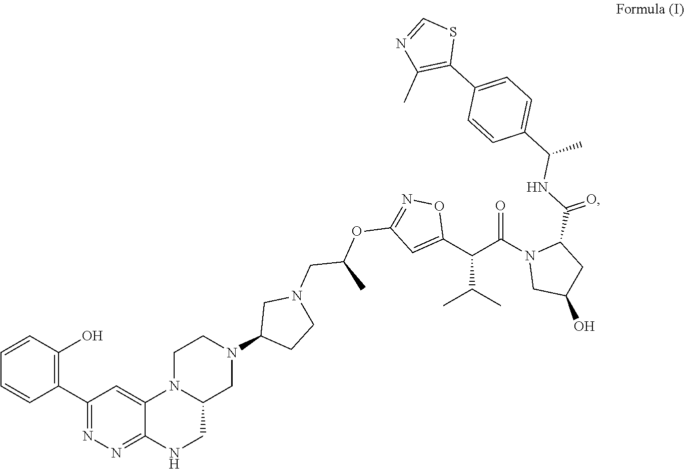 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof; and
(b) a taxane anticancer agent.

Methods of making the compound of Formula (I) are known in the art, and include those set forth in WO2021252666.

In some embodiments of the disclosed methods, the compound of Formula (I), or a pharmaceutically acceptable salt thereof, and the taxane anticancer agent are administered concurrently or sequentially.

In some embodiments of the disclosed methods, the compound of Formula (I), or a pharmaceutically acceptable salt thereof, and the taxane anticancer agent are administered concurrently.

In other embodiments of the disclosed methods, the compound of Formula (I), or a pharmaceutically acceptable salt thereof, and the taxane anticancer agent are administered sequentially.

In some aspects, the subject is administered the compound of Formula (I), or a pharmaceutically acceptable salt of a compound of Formula (I) (on a Formula (I) basis), in an amount of about 20 mg-about 1000 mg per dose, such as, for example, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg per dose.

In some embodiments, the subject is administered the compound of Formula (I), or a pharmaceutically acceptable salt of a compound of Formula (I) (on a Formula (I) basis), in an amount of about 20 mg-about 800 mg per dose.

In some aspects, the compound of Formula (I), or pharmaceutically acceptable salt of a compound of Formula (I), is administered intravenously.

In some embodiments, the compound of Formula (I), or pharmaceutically acceptable salt of a compound of Formula (I), is administered intravenously once per week for 3 weeks.

In some embodiments, the compound of Formula (I), or pharmaceutically acceptable salt of a compound of Formula (I), is administered intravenously in cycles, wherein each cycle comprises administering the compound of Formula (I), or pharmaceutically acceptable salt of a compound of Formula (I), once per week for 3 weeks.

In some aspects of the disclosed methods, the compound of Formula (I), or pharmaceutically acceptable salt of a compound of Formula (I), is administered orally.

In some embodiments, the compound of Formula (I), or pharmaceutically acceptable salt of a compound of Formula (I), is administered orally once per week for 3 weeks.

In some embodiments, the compound of Formula (I), or pharmaceutically acceptable salt of a compound of Formula (I), is administered orally in cycles, wherein each cycle comprises administering the compound of Formula (I), or pharmaceutically acceptable salt of a compound of Formula (I), once per week for 3 weeks.

In some aspects of the disclosed methods, the subject is administered a compound of Formula (I).

In other aspects of the disclosed methods, the subject is administered a pharmaceutically acceptable salt of a compound of Formula (I).

In some aspects of the disclosed methods, the subject is administered a taxane anticancer agent. Taxane anticancer agents are a class of anticancer drugs that bind to tubulin/microtubules and stabilize microtubules, thereby blocking mitosis and resulting in apoptosis. See, e.g., Ojima I, et al. Taxane anticancer agents: a patent perspective. Expert Opin Ther Pat. 2016; 26(1): 1-20. doi: 10.1517/13543776.2016.1111872. Exemplary taxane anticancer agents include paclitaxel, docetaxel, cabazitaxel, and nab-paclitaxel. Other taxane anticancer agents include larotaxel, milataxel, ortataxel, and tesetaxel, BMS-184476, docosahexaenoic acid (DHA)-paclitaxel (“Taxoprexin”), and poly(L-glutamic acid) PG-paclitaxel (“Opaxio”).

In some embodiments, the taxane anticancer agent is docetaxel.

In some embodiments, the taxane anticancer agent is paclitaxel.

In some embodiments, the taxane anticancer agent is cabazitaxel.

In some embodiments, the taxane anticancer agent is nab-paclitaxel.

In some aspects of the disclosed methods, the taxane anticancer agent is administered intravenously.

In some aspects of the disclosed methods, the taxane anticancer agent is administered in an amount of about 10 mg/m² to about 300 mg/m².

In some aspects of the disclosed methods, the taxane anticancer agent is administered in an amount of about 10 mg/m² to about 300 mg/m² per dose such as, for example, about 10 mg/m², about 11 mg/m², about 12 mg/m², about 13 mg/m², about 14 mg/m², about 15 mg/m², about 16 mg/m², about 17 mg/m², about 18 mg/m², about 19 mg/m², about 20 mg/m², about 21 mg/m², about 22 mg/m², about 23 mg/m², about 24 mg/m², about 25 mg/m², about 26 mg/m², about 27 mg/m², about 28 mg/m², about 29 mg/m², about 30 mg/m², about 31 mg/m², about 32 mg/m², about 33 mg/m², about 34 mg/m², about 35 mg/m², about 36 mg/m², about 37 mg/m², about 38 mg/m², about 39 mg/m², about 40 mg/m², about 41 mg/m², about 42 mg/m², about 43 mg/m², about 44 mg/m², about 45 mg/m², about 46 mg/m², about 47 mg/m², about 48 mg/m², about 49 mg/m², about 50 mg/m², about 51 mg/m², about 52 mg/m², about 53 mg/m², about 54 mg/m², about 55 mg/m², about 56 mg/m², about 57 mg/m², about 58 mg/m², about 59 mg/m², about 60 mg/m², about 61 mg/m², about 62 mg/m², about 63 mg/m², about 64 mg/m², about 65 mg/m², about 66 mg/m², about 67 mg/m², about 68 mg/m², about 69 mg/m², about 70 mg/m², about 71 mg/m², about 72 mg/m², about 73 mg/m², about 74 mg/m², about 75 mg/m², about 76 mg/m², about 77 mg/m², about 78 mg/m², about 79 mg/m², about 80 mg/m², about 81 mg/m², about 82 mg/m², about 83 mg/m², about 84 mg/m², about 85 mg/m², about 86 mg/m², about 87 mg/m², about 88 mg/m², about 89 mg/m², about 90 mg/m², about 91 mg/m², about 92 mg/m², about 93 mg/m², about 94 mg/m², about 95 mg/m², about 96 mg/m², about 97 mg/m², about 98 mg/m², about 99 mg/m², about 100 mg/m², about 101 mg/m², about 102 mg/m², about 103 mg/m², about 104 mg/m², about 105 mg/m², about 106 mg/m², about 107 mg/m², about 108 mg/m², about 109 mg/m², about 110 mg/m², about 111 mg/m², about 112 mg/m², about 113 mg/m², about 114 mg/m², about 115 mg/m², about 116 mg/m², about 117 mg/m², about 118 mg/m², about 119 mg/m², about 120 mg/m², about 121 mg/m², about 122 mg/m², about 123 mg/m², about 124 mg/m², about 125 mg/m², about 126 mg/m², about 127 mg/m², about 128 mg/m², about 129 mg/m², about 130 mg/m², about 131 mg/m², about 132 mg/m², about 133 mg/m², about 134 mg/m², about 135 mg/m², about 136 mg/m², about 137 mg/m², about 138 mg/m², about 139 mg/m², about 140 mg/m², about 141 mg/m², about 142 mg/m², about 143 mg/m², about 144 mg/m², about 145 mg/m², about 146 mg/m², about 147 mg/m², about 148 mg/m², about 149 mg/m², about 150 mg/m², about 151 mg/m², about 152 mg/m², about 153 mg/m², about 154 mg/m², about 155 mg/m², about 156 mg/m², about 157 mg/m², about 158 mg/m², about 159 mg/m², about 160 mg/m², about 161 mg/m², about 162 mg/m², about 163 mg/m², about 164 mg/m², about 165 mg/m², about 166 mg/m², about 167 mg/m², about 168 mg/m², about 169 mg/m², about 170 mg/m², about 171 mg/m², about 172 mg/m², about 173 mg/m², about 174 mg/m², about 175 mg/m², about 176 mg/m², about 177 mg/m², about 178 mg/m², about 179 mg/m², about 180 mg/m², about 181 mg/m², about 182 mg/m², about 183 mg/m², about 184 mg/m², about 185 mg/m², about 186 mg/m², about 187 mg/m², about 188 mg/m², about 189 mg/m², about 190 mg/m², about 191 mg/m², about 192 mg/m², about 193 mg/m², about 194 mg/m², about 195 mg/m², about 196 mg/m², about 197 mg/m², about 198 mg/m², about 199 mg/m², about 200 mg/m², about 201 mg/m², about 202 mg/m², about 203 mg/m², about 204 mg/m², about 205 mg/m², about 206 mg/m², about 207 mg/m², about 208 mg/m², about 209 mg/m², about 210 mg/m², about 211 mg/m², about 212 mg/m², about 213 mg/m², about 214 mg/m², about 215 mg/m², about 216 mg/m², about 217 mg/m², about 218 mg/m², about 219 mg/m², about 220 mg/m², about 221 mg/m², about 222 mg/m², about 223 mg/m², about 224 mg/m², about 225 mg/m², about 226 mg/m², about 227 mg/m², about 228 mg/m², about 229 mg/m², about 230 mg/m², about 231 mg/m², about 232 mg/m², about 233 mg/m², about 234 mg/m², about 235 mg/m², about 236 mg/m², about 237 mg/m², about 238 mg/m², about 239 mg/m², about 240 mg/m², about 241 mg/m², about 242 mg/m², about 243 mg/m², about 244 mg/m², about 245 mg/m², about 246 mg/m², about 247 mg/m², about 248 mg/m², about 249 mg/m², about 250 mg/m², about 251 mg/m², about 252 mg/m², about 253 mg/m², about 254 mg/m², about 255 mg/m², about 256 mg/m², about 257 mg/m², about 258 mg/m², about 259 mg/m², about 260 mg/m², about 261 mg/m², about 262 mg/m², about 263 mg/m², about 264 mg/m², about 265 mg/m², about 266 mg/m², about 267 mg/m², about 268 mg/m², about 269 mg/m², about 270 mg/m², about 271 mg/m², about 272 mg/m², about 273 mg/m², about 274 mg/m², about 275 mg/m², about 276 mg/m², about 277 mg/m², about 278 mg/m², about 279 mg/m², about 280 mg/m², about 281 mg/m², about 282 mg/m², about 283 mg/m², about 284 mg/m², about 285 mg/m², about 286 mg/m², about 287 mg/m², about 288 mg/m², about 289 mg/m², about 290 mg/m², about 291 mg/m², about 292 mg/m², about 293 mg/m², about 294 mg/m², about 295 mg/m², about 296 mg/m², about 297 mg/m², about 298 mg/m², about 299 mg/m², or about 300 mg/m² per dose. When the anticancer agent is nab-paclitaxel, the administered taxane anticancer agent is in an amount defined on a paclitaxel basis.

In some embodiments, the taxane anticancer agent is administered in an amount of about 60 mg/m² to about 100 mg/m² per dose.

In some embodiments, the taxane anticancer agent is administered in an amount of about 25 mg/m² per dose.

In some embodiments, the taxane anticancer agent is administered in an amount of about 135 mg/m² to about 175 mg/m² per dose.

In some embodiments, the taxane anticancer agent is administered in an amount of about 125 mg/m² to about 260 mg/m² per dose.

In some embodiments in which the taxane anticancer agent is docetaxel, the docetaxel is administered in accordance with the December 2013 Revision (Sanofi-aventis) of the TAXOTERE (docetaxel) Injection Concentrate, Intravenous Infusion (IV) prescribing information.

In some embodiments in which the taxane anticancer agent is paclitaxel, the paclitaxel is administered in accordance with the April 2011 Revision (Bristol-Myers Squibb) of the TAXOL® (paclitaxel) INJECTION prescribing information.

In some embodiments in which the taxane anticancer agent is carbitaxel, the carbitaxel is administered in accordance with the June 2010 Revision (Sanofi-aventis) of the JEVTANA (cabazitaxel) Injection prescribing information.

In some embodiments in which the taxane anticancer agent is nab-paclitexael, the nab-paclitaxel is administered in accordance with the October 2022 Revision (Bristol-Myers Squibb) of the ABRAXANE® for Injectable Suspension (paclitaxel protein-bound particles for injectable suspension) (albumin-bound), for intravenous use prescribing information.

In some aspects of the disclosed methods, the taxane anticancer agent is administered by intravenous infusion over a period of about 0.5-about 24 hours, such as, for example, about 0.5 hr, about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, or about 24 hr.

In some embodiments, the taxane anticancer agent is administered by intravenous infusion over about 1 hour.

In some embodiments, the taxane anticancer agent is administered by intravenous infusion over about 3 hours.

In some embodiments, the taxane anticancer agent is administered by intravenous infusion over about 6 hours.

In some aspects of the disclosed methods, the taxane anticancer agent is administered by intravenous infusion over a period of about 0.5 hr-about 24 hours once every three weeks, such as, for example, about 0.5 hr, about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, or about 24 hr once every three weeks.

In some embodiments, the taxane anticancer agent is administered by intravenous infusion over about 1 hour once every three weeks.

In some embodiments, the taxane anticancer agent is administered by intravenous infusion over about 3 hours once every three weeks.

In some embodiments, the taxane anticancer agent is administered by intravenous infusion over about 6 hours once every three weeks.

In some aspects of the disclosed methods, the taxane anticancer agent is administered by intravenous infusion over a period of 0.5-24 hours on days 1, 8, and 15 of each 28 day cycle, such as, for example, about 0.5 hr, about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, or about 24 hr on days 1, 8, and 15 of each 28 day cycle.

In some embodiments, the taxane anticancer agent is administered by intravenous infusion over about 0.5 hour on days 1, 8, and 15 of each 28 day cycle.

In some aspects, the treatment regimen of the disclosure further comprise an additional therapeutic agent. Exemplary additional therapeutic agents include, for example, other anticancer agents (e.g., gemcitabine, doxorubicin, cyclophosphamide, cisplatin, carboplatin, fluorouracil), corticosteroids (e.g., prednisone or dexamethasone), antihistamines (e.g., dexchloropheniramine or diphenylhydramine), H₂ antagonist (e.g., ranitidine), and antiemetics.

In some aspects, the methods of the discloser are directed to treating cancer in a subject. In some embodiments, the cancer is one or more of include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

In other embodiments, the cancer is one or more of breast cancer, non-small cell lung cancer, lung squamous cell carcinoma, uterine/endometrio adenocarcinoma, ovarian cancer, lung adenocarcinoma, bladder cancer, skin cancer, hormone refractory prostate cancer, colon adenocarcinoma, gastric adenocarcinoma, pancreatic cancer, AIDS-related Kaposi's sarcoma, or squamous cell carcinoma of the head and neck cancer.

In some embodiments, the cancer is breast cancer or prostate cancer.

In some embodiments, the cancer is breast cancer.

In some embodiments, the cancer is metastatic breast cancer.

In some embodiments, the cancer is prostate cancer.

In some embodiments, the cancer is hormone-refractory metastatic prostate cancer.

In some embodiments, the cancer is non-small cell lung cancer.

In some embodiments, the cancer is adenocarcinoma of the pancreas.

In some embodiments of the disclosed methods, the cancer is a SMARCA4 deficient cancer. As used herein, the term “SMARCA4 deficient” means that the cancer either lacks a SMARCA4 gene, or has a mutated SMARCA4 gene that encodes a fully- or partially-non-functional SMARCA4 protein.

In some embodiments of the disclosed methods, the cancer is not a SMARCA4 deficient cancer.

In some aspects, the methods of the disclosure comprise administering to the subject, in addition to the disclosed treatment regimen, an additional therapeutic agent. Exemplary additional therapeutic agents that may be administered with the disclosed treatment regimen include cyclophosphamide, cisplatin, doxorubicin, prednisone, and fluorouracil.

In some aspects of the methods of the disclosure, administration of the disclosed treatment regimen results in greater tumor growth inhibition than results from administration of either Formula (I) alone or the taxane anticancer agent alone. As used herein, tumor growth inhibition is calculated as follows: Mean % Inhibition=(mean(C)−mean(T))/mean(C)*100%, wherein C is the tumor volume in the control arm, and T is the tumor volume in the treatment arm.

In some aspects of the methods of the disclosure, administration of the disclosed treatment regimen results in at least 40% tumor growth inhibition, such as, for example, at least 40%, at least 45%, at least 50%, at least 55%, at least 60%, at least 65%, at least 70%, at least 75%, at least 80%, at least 85%, at least 90%, or at least 95% tumor growth inhibition, relative to no treatment.

In some aspects of the methods of the disclosure, the cancer exhibits a complete response (CR) or a partial response (PR) to the administration of the treatment regimen, as evaluated by the Response Evaluation Criteria in Solid Tumors (RECIST) v1.1 criteria.

In some embodiments, the cancer exhibits a complete response (CR) to the administration of the treatment regimen, as evaluated by the Response Evaluation Criteria in Solid Tumors (RECIST) v1.1 criteria.

In other embodiments, the cancer exhibits a partial response (PR) to the administration of the treatment regimen, as evaluated by the Response Evaluation Criteria in Solid Tumors (RECIST) v1.1 criteria.

The disclosure is also directed to the following aspects:

Aspect 1. A method of treating cancer in a subject in need thereof, comprising administering to the subject a therapeutically effective amount of a SMARCA2 inhibitor.

Aspect 2. The method of aspect 1, wherein the SMARCA2 inhibitor comprises Compound I, or a pharmaceutically acceptable salt thereof.

Aspect 3. The method of any one of the preceding aspects, wherein the SMARCA2 inhibitor is administered using a dosing regimen and schedule disclosed herein.

Aspect 4. The method of any one of the preceding aspects, wherein the cancer comprises one or more tumors with loss of SMARCA4.

Aspect 5. The method of any one of the preceding aspects, wherein the cancer comprises one or more tumors with mutated SMARCA4.

### EXAMPLES

### Example 1

The purpose of this study is to evaluate the effect of Formula (I)+docetaxel combination on cancer cell cycle and proliferation. SMARCA4 deficient cancer cell lines were utilized to demonstrate whether the Formula (I) combination with docetaxel led to higher cell viability inhibition and enhanced cell cycle arrest compared to each single agent alone.

### Materials

Formula (I)
   Formula (I) was stored in powder form at −20° C. The compound was dissolved in 100% DMSO to make a 10 mM stock. The dissolved DMSO solution was stored at −20° C.
Docetaxel (Selleckchem Cat #: S1148)
CellTiter-Glo (Promega, Cat #: G7570).
Click-iT EdU Pacific Blue Flow Cytometry Assay Kit (Invitrogen, Cat #: C10418)
DraQ5 (Bio legend, Cat #: 424101)

### Procedures

### Cell Lines

All cell lines used in this study were obtained from ATCC: NCI-H838, NCI-H2030. All cell lines were cultured and maintained according to the conditions specified by the respective cell banks. All of the cells were maintained in RPMI1640 media supplemented with 10% FBS and 1% penicillin/streptomycin and grown at 37° C. and 5% CO₂ in an incubator.

### Cell Titer-Glo Proliferation Assay

The cells were maintained in RPMI1640 media supplemented with 10% FBS and Pen/Strep in a humidified CO₂ incubator at 37° C. The harvested cells were re-suspended at 5 to 10×10³ cells/ml and 100 μl of the cell suspension was dispensed to each well of 384-well plates (solid white, ThermoFisher) by multichannel pipette. After overnight incubation, compounds were dispensed by D300e Tecan digital dispenser. The cells were incubated at 37° C. in 5% CO₂ for 5-7 days. Cell viability was measured by Cell-Titer Glo (Promega). RLU was detected by Envision plate reader (PerkinElmer). Combination synergy was determined using the web-based tool SynergyFinder to produce ZIP scores. See Ianevski, A., A. K. Giri, and T. Aittokallio, SynergyFinder 3.0: an interactive analysis and consensus interpretation of multi-drug synergies across multiple samples. Nucleic Acids Research, 2022. 50(W1): p. W739-W743.

### Cell Cycle Analysis

Cells were seeded, incubated overnight, and dosed with drug treatments the next day. 2 hours before collection, cells were incubated with 10 μM EdU at 37 C then harvested using trypsin EDTA and washed with PBS/1% BSA. 4% PFA was added to the cells followed by a 15 minute incubation at RT. Cell were washed again with PBS/1% BSA and resuspended in 1X Click-iT® saponin-based permeabilization reagent for 15-minutes. Following centrifugation, supernatant was removed, and cells were incubated for 30 mins at RT, protected from light, in Click-iT® reaction cocktail, which was prepared according to manufacturer's instructions. Cells were then washed once with 0.01% Triton in PBS and resuspended in PBS. DRAQ5™ was added to cells at 1:200 dilution, incubated at room temperature, preventing exposure to light, for 30-60 min. Lastly, cells were washed with PBS/1% BSA and resuspended in 400 μl PBS/1% BSA. EdU with Pacific Blue™ azide and was detected using 405 nm excitation with a violet emission filter (450/50 nm) on a flow cytometer and analyzed using FlowJo software.

### Data Analysis

Appropriate read out parameters in each assay described above were normalized to signals from the DMSO control treated cells. Percent cell viability was plotted against concentration of test compounds. The half-maximal inhibitory values (IC₅₀) were determined using a GraphPad Prism software.

### Results

The Formula (I)+docetaxel combination led to synergistic inhibitory cell viability effects in SMARCA4-deficient cell lines.

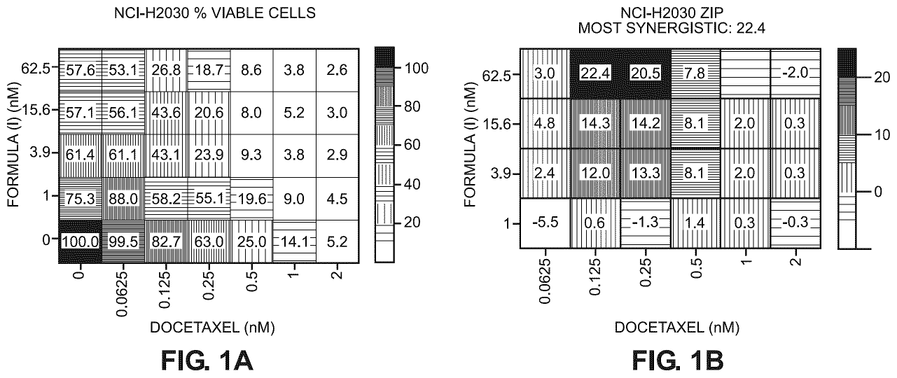

The combination effects of Formula (I) and docetaxel on NSCLC tumor cell viability was evaluated in SMARCA4 deficient lung cancer cell lines by CTG assay. In the SMARCA4-low expressing NSCLC cell line NCI-H2030, cells were dosed with Formula (I) and docetaxel in a synergy matrix using the D300e Digital Dispenser (Tecan) with DMSO volume normalization. Following the treatment incubation period, the fluorescent intensity of each well was normalized to the average of the DMSO control wells on the same plate to calculate relative cell viability values (FIG. 1A). For synergy analysis, relative cell viability measurements from quadruplicate wells were averaged and analyzed using the web-based tool SynergyFinder to produce ZIP scores. The combination of Formula (I) and docetaxel demonstrated excellent synergy (FIG. 1B), with ZIP scores larger than 10 indicating that the interaction between two drugs is likely to be synergistic. See Ianevski, A., A. K. Giri, and T. Aittokallio, SynergyFinder 3.0: an interactive analysis and consensus interpretation of multi-drug synergies across multiple samples. Nucleic Acids Research, 2022. 50(W1): p. W739-W743.

Formula (I)+docetaxel combination resulted in enhanced G2/M arrest in a SMARCA4-deficient human lung cancer cell line

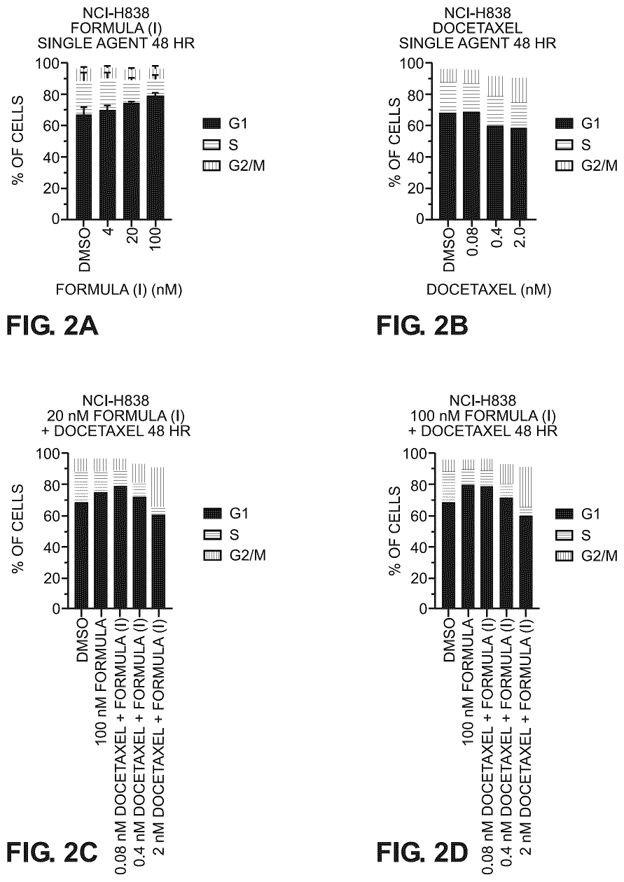

The combination effects of Formula (I) and docetaxel on the cell cycle was analyzed in a SMARCA4-deficient NSCLC lung cancer cell line by flow cytometry using DraQ5 and Click-iT EdU Pacific Blue. In the SMARCA4-del NSCLC cell line NCI-H838, cells were dosed with single agent Formula (I) (FIG. 2A), single agent docetaxel (FIG. 2B), and Formula (I)+docetaxel combinations with a Formula (I) relative low dose (FIG. 2C) and a Formula (I) relative high dose (FIG. 2D) for 48 hrs. Following this, cells were incubated with EdU, fixed permeabilized and stained with DRAQ5™ prior to cell cycle analysis. Single agent Formula (I) treatment induced a dose dependent G1 arrest signal (FIG. 2A), consistent with selective SMARCA2 degrader treatment in SMARCA4-deficient NSCLC cell lines. See Cantley, J., et al., Selective PROTAC-mediated degradation of SMARCA2 is efficacious in SMARCA4 mutant cancers. Nature Communications, 2022. 13(1): p. 6814. In contrast, single agent docetaxel treatment induced a dose dependent G2/M arrest signal (FIG. 2B), consistent with Han, T. D., et al. Docetaxel enhances apoptosis and G2/M cell cycle arrest by suppressing mitogen-activated protein kinase signaling in human renal clear cell carcinoma. Genet Mol Res, 2016. 15(1); and Morse, D. L., et al. Docetaxel induces cell death through mitotic catastrophe in human breast cancer cells. Molecular Cancer Therapeutics, 2005. 4(10): p. 1495-1504. Interestingly, when Formula (I) was combined with docetaxel, there was a pronounced increase in the % cell population arrested at G2/M vs docetaxel single agent treatment (FIGS. 2C-D).

These results demonstrate that the Formula (I)+docetaxel combination led to synergistic inhibitory cell viability effects as well as enhanced G2/M arrest in SMARCA4-deficient human lung cancer cell lines.

### Example 2

A study was conducted to evaluate the anti-tumor efficacy of the Formula (I)+docetaxel combination in SMARCA4 deficient and SMARCA4 WT human lung cancer xenogtaft models in mice. Specific objectives included:
1. To determine whether the Formula (I)+docetaxel combination in SMARCA4 deficient lung cancer models in mice results in deeper tumor growth inhibition (TGI) compared to each single agent alone.
2. To determine whether the Formula (I)+docetaxel combination is well-tolerated in mice.
3. To examine whether repeat administration of docetaxel has any effect on Formula (I) target (SMARCA2 protein levels).
4. To examine any adverse drug-drug interactions (DDIs) between Formula (I) and docetaxel in mice.

### Materials

Formula (I)

Formula (I) was stored in powder form at −20° C.
Docetaxel (Jiangsu Hengrui Medicine CO., LTD)
   Docetaxel was stored in solution form at 2-8° C.
Nab-paclitaxel (CSPC Pharmaceutical Group Co., Ltd)
   Nab-paclitaxel was stored in solution form at 2-8° C.

### Experimental Procedures

### NCI-H838 Tumor Bearing Mouse Model

The NCI-H838-#5 tumor cells were maintained in vitro with RPMI1640 medium supplemented with 10% fetal bovine serum at 37° C. in an atmosphere of 5% CO₂ in air. The cells in exponential growth phase were harvested and quantitated by cell counter before tumor inoculation. NOD-SCID female mice were inoculated subcutaneously in the right front flank region with NCI-H838-#5 tumor cells (5×10⁶) in 0.1 ml of PBS mixed with Matrigel (1:1) for tumor development. The randomization was performed when the mean tumor size reaches approximately 100-150 mm³. Tumor volumes were measured every other day after randomization in two dimensions using a caliper, and the volume will be expressed in mm³ using the formula: “V=(L×W×W)/2, where V is tumor volume, L is tumor length (the longest tumor dimension) and W is tumor width (the longest tumor dimension perpendicular to L).

### A549 Tumor Bearing Mouse Model

The A549 tumor cells were maintained in vitro with Ham's F12K medium supplemented with 10% fetal bovine serum at 37° C. in an atmosphere of 5% CO₂ in air. The cells in exponential growth phase were harvested and quantitated by cell counter before tumor inoculation. BALB/c nude female mice were inoculated subcutaneously in the right front flank region with A549 tumor cells (5×10⁶) in 0.1 ml of PBS for tumor development. The randomization was performed when the mean tumor size reaches approximately 100-150 mm³. Tumor volumes were measured every other day after randomization in two dimensions using a caliper, and the volume will be expressed in mm³ using the formula: “V=(L×W×W)/2, where V is tumor volume, L is tumor length (the longest tumor dimension) and W is tumor width (the longest tumor dimension perpendicular to L).

### Calu-6 Tumor Bearing Mouse Model

The Calu-6 tumor cells were maintained in vitro with MEM medium supplemented with 10% fetal bovine serum at 37° C. in an atmosphere of 5% CO₂ in air. The cells in exponential growth phase were harvested and quantitated by cell counter before tumor inoculation. BALB/c nude female mice were inoculated subcutaneously in the right front flank region with Calu-6 tumor cells (5×10⁶) in 0.1 ml of PBS for tumor development. The randomization was performed when the mean tumor size reaches approximately 100-150 mm³. Tumor volumes were measured every other day after randomization in two dimensions using a caliper, and the volume will be expressed in mm³ using the formula: “V=(L×W×W)/2, where V is tumor volume, L is tumor length (the longest tumor dimension) and W is tumor width (the longest tumor dimension perpendicular to L).

### Western Blot

SMARCA2 protein expression was measured by semi-quantitative western blot following extraction of protein from frozen tumor tissue. Up to 50 mg of frozen tumor tissue was measured and placed into 0.5 mL conical tubes containing 0.5 mm ceramic beads and 400 μL RIPA buffer containing 1× protease and phosphatase inhibitors (ThermoFisher Scientific, A32959) and PMSF (Cell Signaling Technology, #8553). Tissues were homogenized with a Bead Ruptor Elite at a speed of 5 m/sec for 25 seconds and centrifuged at 12,000 rpm for 3 min at 4° C. Milling and centrifugation steps were performed two additional times before transferring supernatant to UltraFree 0.45 μm 0.5 mL filter columns (Millipore Sigma, #UFC30HV) and centrifuging at 12,000 rpm for 1 min. Flow through lysate was quantified by BCA assay, diluted with 1 volume of RIPA lysis buffer, and mixed with Laemmli sample buffer. Lysates were boiled at 95° C. for 5 min and 30 μg of protein was loaded in 4-20% Criterion TGX Precast Midi Protein Gels (BioRad, #5671095). Protein was transferred to PVDF membranes by Trans-Blot Turbo system (Bio-Rad) and probed with Actin (Signma, #A2228) and SMARCA2 (Cell Signaling Technology, #11966) primary antibodies at a 1:1000 dilution and IRDye 800CW or IRDye 680RD secondary antibodies (LICOR, antibody, #926-68070) in iBind automated western system (ThermoFisher Scientific). The stained blots were scanned by LiCOR Odyssey CLx imaging system to acquire integrated intensities at 700 nm and 800 nm channel and analyzed using Image Studio software v5.2.

### Data Analysis

Tumor volumes at the end of studies were evaluated by the Mann-Whitney test (unpaired, nonparametric test). * P<0.05 ** P<0.05, *** P<0.001 versus vehicle are shown in graphs.

### Results

Formula (I) combination with docetaxel resulted in deeper tumor growth inhibition (TGI) compared to each single agent alone in NCI-H838 SMARCA4 deficient tumors at well tolerated doses

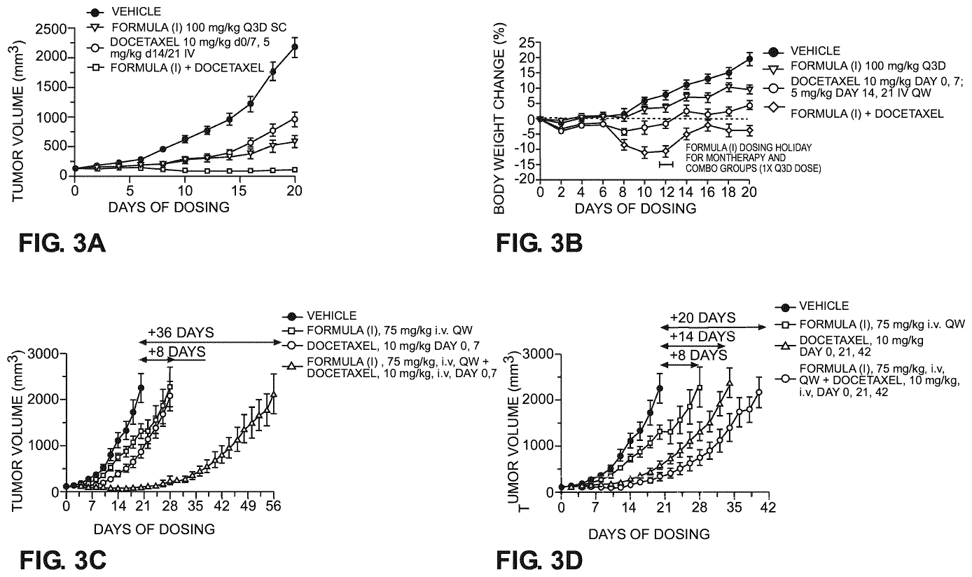

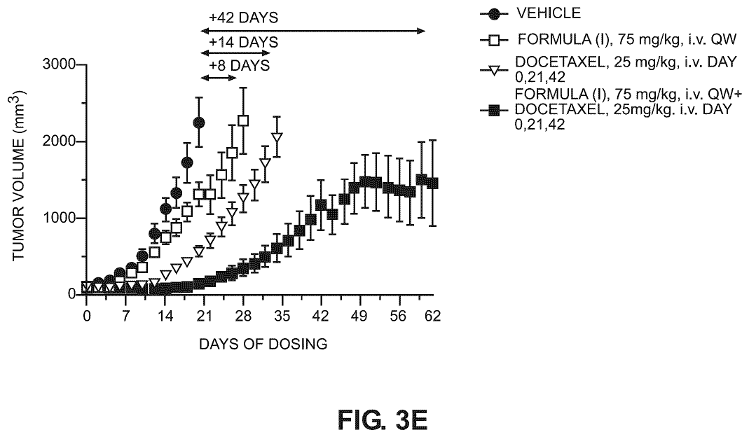

The efficacy of Formula (I) and docetaxel on tumor growth inhibition was evaluated in SMARCA4 deficient lung cancer models in mice. For the NCI-H838 CDX model, mice were inoculated subcutaneously with the NCI-H838 tumor cells (5×10⁶). Formula (I) administered subcutaneously (s.c.) every third day (Q3D) at 100 mg/kg significantly inhibited the growth of NCI-H838 tumors with a TGI of 73% (FIG. 3A and Table 2). Once weekly (QW) intravenous (i.v.) administration of docetaxel at 10 mg/kg on days 0,7 followed by reduction to 5 mg/kg day on days 14, 21 also showed significant tumor growth inhibition in this model with a TGI of 55% (FIG. 3A and Table 2). The combination of Formula (I) (100 mg/kg s.c. Q3D) and docetaxel (5-10 mg/kg i.v. QW) resulted in a significantly higher TGI compared to each single agent alone (TGI vs Vehicle 95%). The docetaxel single agent dose decrease was matched in the combination group. Administration of Formula (I) and docetaxel single agents was well tolerated on these dosing regimens, with no significant morbidity or body weight loss observed (FIG. 3B). The combination of Formula (I) and docetaxel required one Formula (I) dosing holiday (this was matched in the Formula (I) single agent group) on day 12 following moderate weight loss. Mice body weights recovered following this, and no further dosing holidays were given (FIG. 3B). The efficacy of Formula (I) and docetaxel on tumor growth inhibition was further evaluated at different doses and dosing schedule in the NCI-H838 CDX model in mice. Formula (I) weekly i.v. dosing at 75 mg/kg significantly delayed the tumor growth of NCI-H838 in combination with docetaxel 10 mg/kg i.v. administrated on day 0 and day 7 (FIG. 3C), docetaxel 10 mg/kg i.v. administrated on day 0, 21, and 42 (FIG. 3D), or docetaxel 25 mg/kg i.v. administrated on day 0, 21 and 42 (FIG. 3E), compared to each single agent alone.

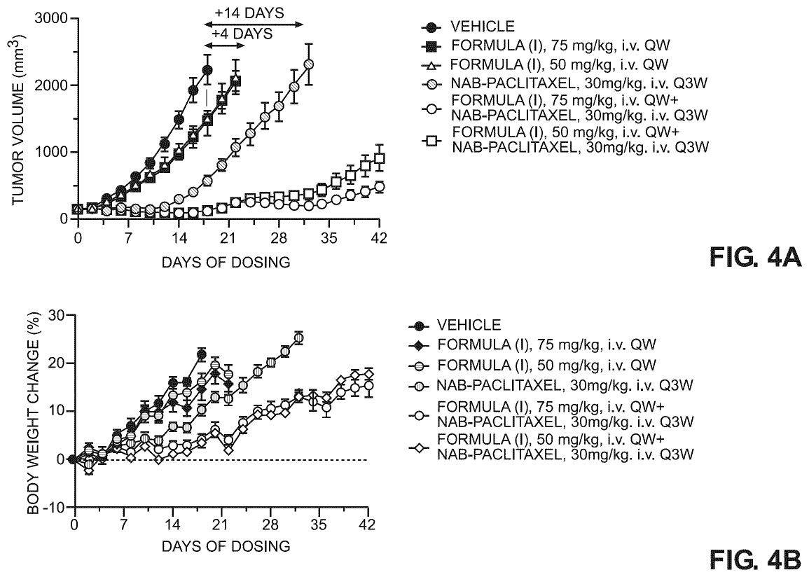

The efficacy of Formula (I) and nab-paclitaxel on tumor growth inhibition was evaluated in SMARCA4 deficient lung cancer models in mice. For the NCI-H838 CDX model, mice were inoculated subcutaneously with the NCI-H838 tumor cells (5×10⁶). Formula (I) weekly i.v. dosing at 50 mg/kg or 75 mg/kg significantly delayed the tumor growth of NCI-H838 in combination with nab-paclitaxel 30 mg/kg i.v. administrated every three week (FIG. 4A). Administration of Formula (I) and nab-paclitaxel single agents was well tolerated on these dosing regimens, with no significant morbidity or body weight loss observed (FIG. 4B).

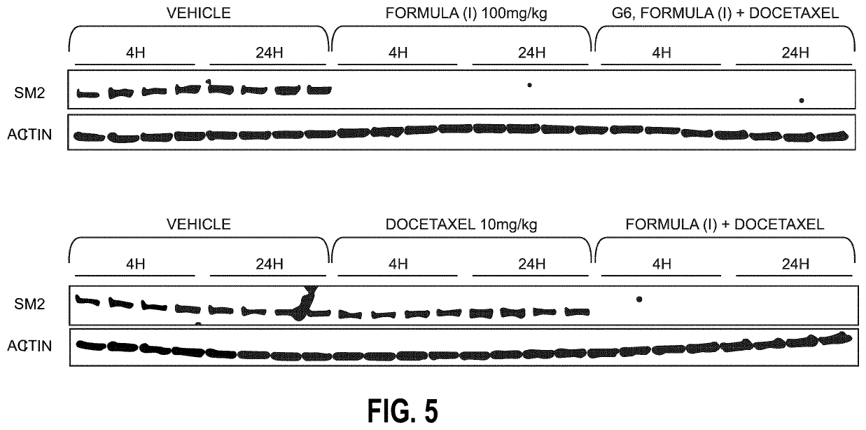

Tumor tissues collected at the end of efficacy studies were analyzed for SMARCA2 protein levels by semi-quantitative western blot. Repeat administration of Formula (I) in these studies significantly decreased tumor SMARCA2 protein levels in SMARCA4 deficient cancer cells relative to vehicle treated mice (FIG. 5). Conversely, repeat administration of docetaxel (5-10 mg/kg i.v. QW) had no effect on SMARCA2 protein levels in SMARCA4 deficient cancer cells relative to vehicle treated mice (FIG. 5).

Formula (I) combination with docetaxel resulted in deeper tumor growth inhibition (TGI) compared to each single agent alone in A549 SMARCA4 deficient tumors at well tolerated doses

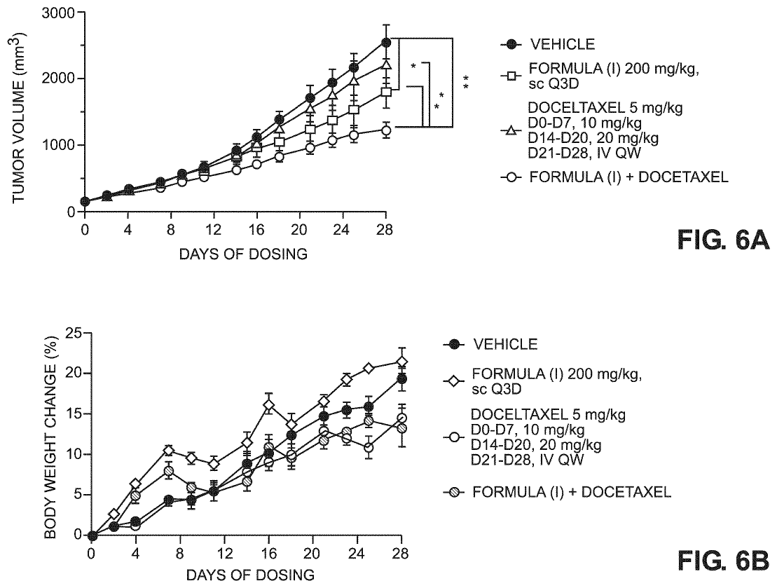

The efficacy of Formula (I) and docetaxel on tumor growth inhibition was also evaluated in another SMARCA4 deficient non-small cell lung cancer model, A549. For the A549 CDX model, mice were inoculated c with A549 tumor cells (5×10⁶). Formula (I) administered s.c. every third day (Q3D) at 200 mg/kg significantly inhibited the growth of A549 tumors with a TGI of 32% (FIG. 6A and Table 2). i.v. administration of docetaxel at 5 mg/kg on days 0, 7, 10 followed by increases to 10 mg/kg on days 14 and 20 mg/kg on Days 21 and 28 also showed significant tumor growth inhibition in this model with a TGI of 13% (FIG. 6A and Table 2). The combination of Formula (I) (100 mg/kg s.c. Q3D) and docetaxel (5-20 mg/kg i.v. QW) resulted in a significantly higher TGI compared to each single agent alone (TGI vs vehicle 55%). The docetaxel single agent dose increases were matched in the combination group. Administration of Formula (I) and docetaxel single agents and Formula (I)+docetaxel combination agents was well tolerated on these dosing regimens, with no significant morbidity or body weight loss observed (FIG. 6B).

The Formula (I) combination with docetaxel resulted in no significant increase in growth inhibition (TGI) compared to each single agent alone in Calu-6 SMARCA4 WT tumors at well tolerated doses

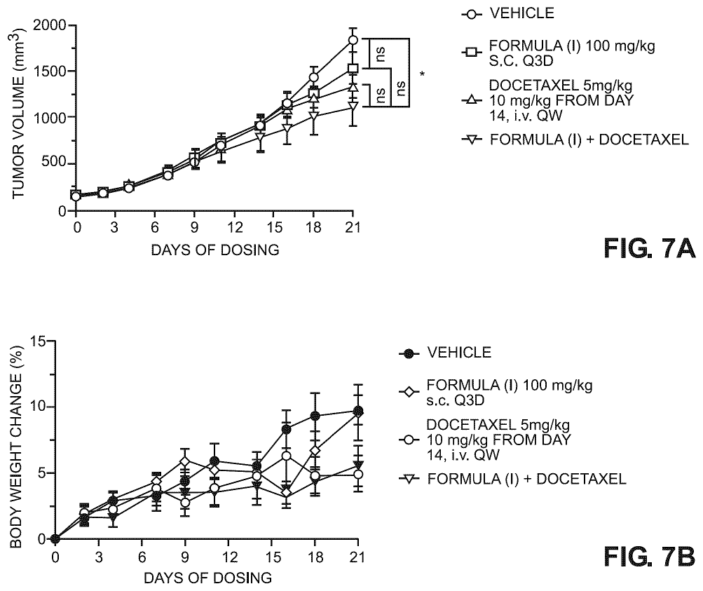

Formula (I) administration at efficacious dosing regimens did not inhibit the growth of SMARCA4 WT Calu-6 lung tumor xenografts in mice. In addition, Formula (I) combination with docetaxel resulted in no significant increase in growth inhibition (TGI) compared to each single agent alone (FIG. 7A). Administration of Formula (I) and docetaxel single agents and Formula (I)+docetaxel combination agents was well tolerated on these dosing regimens, with no significant morbidity or body weight loss observed (FIG. 7B).

A summary of all tested doses, dosing schedules, and anti-tumor efficacy of Formula (I) single agent, docetaxel single agent and Formula (I)+docetaxel combination agents in two SMARCA4 deficient non-small cell lung cancer models and one SMARCA4 WT non-small cell lung cancer models is provided in Table 2.

TABLE 1
Summary of Anti-tumor Efficacy of Formula (I)
in SMARCA4-deficient lung Cancer Models in Mice

| Model (Study | | | | P value |
|---|---|---|---|---|
| report # in | | Tumor size | | (Mann- |
| Appendix) | Group | (mm³)^{a} | TGI^{b} (%) | Whitney test)^{c} |
| NCI-H838 CDX | Vehicle | 2173.91 ± 163.45 | | |
| (E3445-U2301) | | (day 20) | | |
| | Formula (I) 100 | 590.46 ± 102.37 | 73.0% (day | 0.0002 |
| | mg/kg s.c. Q3D | (day 20) | 20) | |
| | Docetaxel 10/5 | 971.86 ± 127.82 | 55.0% (day | 0.0002 |
| | mg/kg i.v. QW | (day 20) | 20) | |
| | Formula (I) 100 | 113.13 ± 22.27 | 95.0% (day | 0.0002 |
| | mg/kg s.c. Q3D + | (day 20) | 20) | |
| | Docetaxel 10/5 | | | |
| | mg/kg i.v. QW | | | |
| A549 CDX | Vehicle | 2,540 ± 262 | | |
| (PRELUDE- | | (day 28) | | |
| 20230703A) | Formula (I) 100 | 1,785 ± 221 | 31.6% (day | 0.041 |
| | mg/kg s.c. Q3D | (day 28) | 28) | |
| | Docetaxel, | 2,223 ± 300 | 13.3% (day | 0.58 |
| | 5/10/20 mg/kg, | (day 28) | 28) | |
| | QW | | | |
| | Formula (I) + | 1,230 ± 122 | 54.89% (day | 0.002 |
| | Docetaxel, 200 + | | 28) | |
| | 5/10/20 mg/kg, | | | |
| | Q3D + QW | | | |
| Calu-6 CDX | Vehicle | 2,049 ± 242 | | |
| (PRELUDE- | | (day 21) | | |
| 20230703) | Formula (I), 100 | 1,535 ± 174 | 27.2% (day | 0.23 |
| | mg/kg, Q3D | (day 21) | 21) | |
| | Docetaxel, 5/10 | 1,338 ± 132 | 37.6% (day | 0.02 |
| | mg/kg, QW | (day 21) | 21) | |
| | Formula (I) + | 1,113 ± 205 | 49.5% (day | 0.02 |
| | Docetaxel, 100 + | (day 21) | 21) | |
| | 5/10 mg/kg, | | | |
| | Q3D + QW | | | |

^{a}Mean ± SEM (Standard error of the means)
^{b}Tumor growth inhibition
^{c}Compared to vehicle (Unpaired non-parametric test, Mann-Whitney test)

These studies indicate that the Formula (I) combination with docetaxel resulted in deeper tumor growth inhibition (TGI) compared to each single agent alone in SMARCA4 deficient lung cancers in vivo, but not SMARCA4 WT lung tumors. Repeat administration of Formula (I) (100-200 mg/kg s.c. Q3D) resulted in complete reduction of tumor SMARCA2 protein levels over a 24 h period providing pharmacodynamic confirmation of the activity of Formula (I) in vivo. Repeat administration of docetaxel (5-20 mg/kg i.v. QW) had no effect on SMARCA2 protein levels and PK analysis revealed no adverse drug-drug interactions (DDIs) between Formula (I) and docetaxel.

When Formula (I) was combined with docetaxel in vitro, synergistic effects were observed in SMARCA4 mutated cancer cell lines (FIG. 1). The combination led to G2/M phase cell cycle arrest (FIG. 2). This combination treatment was further tested in vivo in the NCI-H838 SMARCA4 mutated NSCLC CDX model. When Formula (I) was combined with docetaxel, deeper tumor growth inhibition (TGI) was observed compared to each single agent alone (TGI: Formula (I)=73%, docetaxel=55%, combination=95%). In the A549 model, a second SMARCA4 mutant NSCLC model, Formula (I) also demonstrated enhanced tumor growth inhibition when combined with docetaxel. These combination effects were not observed in a SMARCA4 WT Calu6 tumor model. In all models tested, the combination was well tolerated.

### Example 3

To elucidate the mechanism of action for the combination effects observed in vivo, RNA-sequencing was conducted on SMARCA4-mutated NCI-H838 tumor tissues treated with Formula (I) and/or docetaxel for one week. Gene Set Enrichment Analysis demonstrated that docetaxel monotherapy led to a positive enrichment of E2F targets and G2/M pathways, suggesting cellular stress responses to the treatment. Conversely, Formula (I) counteracted these processes by downregulating E2F targets and G2/M pathways, including genes such as CDC45, RAD51API, TOP2A, and EGF.

Formula (I) in combination with docetaxel synergistically increased the expression of pro-apoptotic genes, including CDKN1A and GADD45A. Furthermore, Formula (I) induced G1 cell cycle arrest in SMARCA4-mutated cancer cell lines, but not in SMARCA4 wild-type cell lines, while docetaxel induced G2/M arrest. Together, these data show that the combination of Formula (I) and docetaxel leads to the disruption of multiple distinct cell cycle checkpoints and results in a more complete cell cycle blockade in SMARCA4-mutated models. Thus, the combination of Formula (I) with NSCLC standard of care chemotherapy agents demonstrates significantly enhanced anti-tumor activities in preclinical models of SMARCA4-mutated NSCLC.

1. A method of treating cancer in a subject in need thereof, comprising administering to the subject a treatment regimen comprising:
(a) a compound of Formula (I): 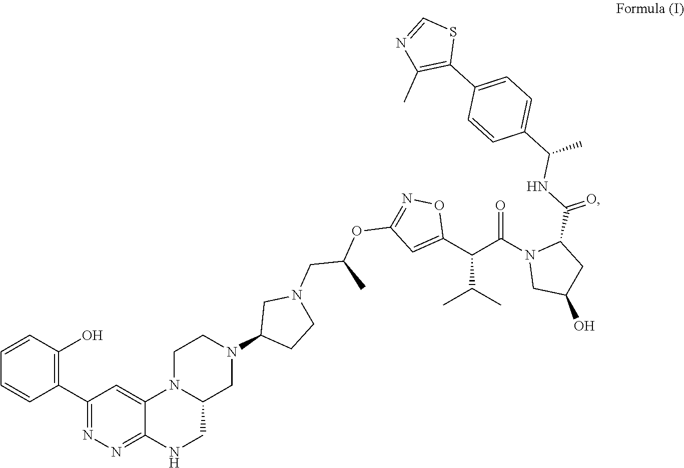 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]
or a pharmaceutically acceptable salt thereof; and
(b) a taxane anticancer agent.

2. The method of claim 1, wherein the compound of Formula (I), or a pharmaceutically acceptable salt thereof, and the taxane anticancer agent are administered concurrently or sequentially.

3. The method of claim 1, wherein the taxane anticancer agent is docetaxel, paclitaxel, nab-paclitaxel, or cabazitaxel.

4. The method of claim 3, wherein the taxane anticancer agent is docetaxel.

5. The method of claim 1, wherein the subject is administered the compound of Formula (I), or a pharmaceutically acceptable salt of a compound of Formula (I) (on a Formula (I) basis), in an amount of about 20 mg-about 1000 mg per dose.

6. The method of claim 1, wherein the compound of Formula (I), or pharmaceutically acceptable salt of a compound of Formula (I), is administered intravenously.

7. The method of claim 1, wherein the compound of Formula (I), or pharmaceutically acceptable salt of a compound of Formula (I), is administered orally.

8. The method of claim 1, wherein the compound of Formula (I), or pharmaceutically acceptable salt of a compound of Formula (I), is administered once per week for 3 weeks.

9. The method of claim 1, wherein the subject is administered a compound of Formula (I).

10. The method of claim 1, wherein the subject is administered a pharmaceutically acceptable salt of a compound of Formula (I).

11. The method of claim 1, wherein the taxane anticancer agent is administered intravenously.

12. The method of claim 1, wherein the taxane anticancer agent is administered in an amount of about 10 mg/m² to about 300 mg/m² per dose.

13. The method of claim 11, wherein the taxane anticancer agent is administered by intravenous infusion over about 0.5 hr-about 3 hours, once every three weeks.

14. The method of claim 1, wherein the treatment regimen further comprises an additional therapeutic agent.

15. The method of claim 14, wherein the additional therapeutic agent is gemcitabine, doxorubicin, cyclophosphamide, cisplatin, carboplatin, fluorouracil, prednisone, dexamethasone, dexchloropheniramine, diphenylhydramine, ranitidine, or an antiemetic.

16. The method of claim 1, wherein the cancer is breast cancer, non-small cell lung cancer, lung squamous cell carcinoma, uterine/endometrio adenocarcinoma, ovarian cancer, lung adenocarcinoma, bladder cancer, skin cancer, hormone refractory prostate cancer, colon adenocarcinoma, gastric adenocarcinoma, pancreatic cancer, AIDS-related Kaposi's sarcoma, or squamous cell carcinoma of the head and neck cancer.

17. The method of claim 1, wherein the cancer is a SMARCA4 deficient cancer.

18. The method of claim 1, wherein administration of the treatment regimen results in greater tumor growth inhibition than results from administration of either Formula (I) alone or the taxane anticancer agent alone.

19. The method of claim 1, wherein administration of the treatment regimen results in at least 40% tumor growth inhibition relative to no treatment.

20. The method of claim 1, wherein the cancer exhibits a complete response (CR) or a partial response (PR) to the administration of the treatment regimen, as evaluated by the Response Evaluation Criteria in Solid Tumors (RECIST) v1.1 criteria.

21. The method of claim 20, wherein the cancer exhibits a complete response (CR) to the administration of the treatment regimen, as evaluated by the Response Evaluation Criteria in Solid Tumors (RECIST) v1.1 criteria.

22. The method of claim 20, wherein the cancer exhibits a partial response (PR) to the administration of the treatment regimen, as evaluated by the Response Evaluation Criteria in Solid Tumors (RECIST) v1.1 criteria.
